(19) United States
(12) Patent Application Publication
(10) Pub. No.: US 2012/0041280 A1
(43) Pub. Date: Feb. 16, 2012

(54) APPARATUS AND METHOD FOR BODY INFORMATION ACQUISITION IN PORTABLE TERMINAL

(75) Inventor: Jong-Min LEE, Incheon (KR)

(73) Assignee: Samsung Electronics Co., Ltd., Gyeonggi-Do (KR)

(21) Appl. No.: 13/210,594

(22) Filed: Aug. 16, 2011

(30) Foreign Application Priority Data
Aug. 16, 2010 (KR) 10-2010-0078701

(51) Int. Cl. A61B 5/0205; A61B 5/021; A61B 5/00

(52) U.S. Cl. 600/301; 600/300; 600/485

(57) ABSTRACT

A method for determining a user's body information in a mobile terminal includes determining whether a user touch is detected on a touch panel, which incorporates a Bio Micro Electric Mechanical System (MEMS) technology, and if so, determining the user's body information which in turn is transmitted to an upper node. Body information analysis results are received from the upper node and displayed subsequently. As a result, a mobile user has an advantage of managing his or her health via on line using a touch panel of a mobile terminal without visiting a hospital separately.

### CLAIM OF PRIORITY

This application claims the benefit under 35 U.S.C. §119(a) of a Korean patent application filed in the Korean Intellectual Property Office on Aug. 16, 2010 and assigned Serial No. 10-2010-0078701, the entire disclosure of which is hereby incorporated by reference.

### BACKGROUND OF THE INVENTION

1. Field of the Invention

The present invention relates to an apparatus and a method for determining biological information using a device that can detect a body state when a user touches a touch screen of a mobile terminal, and managing the user's health based on the detected body information.

2. Description of the Related Art

A recent mobile terminal supports a touch screen that uses a touch panel, which includes a panel and a window that can detect a touch. In operation, when using the mobile terminal, a user directly and physically contacts the window, which is made of acryl or tempered glass material.

However, to date, the window is not used for other purposes or application other than protecting inner parts of the terminal and preventing a reflection thereof.

### SUMMARY OF THE INVENTION

An aspect of the present invention is to provide an apparatus and method for acquiring and detecting a user's health related information via a portable terminal.

Another aspect of the present invention is to provide an apparatus and a method for managing a user's health on line by integrating a nano technology and a Bio Micro Electric Mechanical System (MEMS) technology with a window forming a touch panel and determining the user' body information in a mobile terminal.

In accordance with an aspect of the present invention, a method for determining a user's body information in a mobile terminal is provided. The method includes determining whether a touch input is detected with respect to a touch panel to which a Bio Micro Electric Mechanical System (MEMS) technology has been applied, when the touch is detected, determining the user's body information, transmitting the determined body information to an upper node, receiving body information analysis results from the upper node, and displaying the body information analysis results.

In accordance with another aspect of the present invention, an apparatus of a mobile terminal that determines a user's body information is provided. The apparatus includes a touch panel including a window to which a Bio Micro Electric Mechanical System (MEMS) technology has been applied and detecting a touch input, a pressure sensor, a modem for transmitting/receiving information to/from an upper node, a controller for, upon detecting the touch input on the touch panel, determining the user's body information via the touch panel, transmitting the determined body information to the upper node via the modem, and receiving body information analysis results from the upper node via the modem, and a display unit for displaying the body information analysis results.

In accordance with further another aspect of the present invention, a mobile communication system for performing a user's health management is provided. The system includes a mobile terminal for determining whether an touch input exists with respect to a touch panel to which a Bio Micro Electric Mechanical System (MEMS) technology has been applied, upon detecting the touch input, determining the user's body information, transmitting the determined body information to a server, and upon receiving a response from the server, displaying the response, the server for transmitting the body information transmitted by the mobile terminal to a hospital server, and transmitting a response transmitted by the hospital server to the mobile terminal, and the hospital server for analyzing the user's body information transmitted by the server, and when an abnormal part exists as a result of the body information analysis, transmitting an analysis result including the abnormal part and a processing guide for the abnormal part to the server.

Other aspects, advantages and salient features of the invention will become apparent to those skilled in the art from the following detailed description, which, taken in conjunction with the annexed drawings, discloses exemplary embodiments of the invention.

### BRIEF DESCRIPTION OF THE DRAWINGS

The above and other aspects, features and advantages of certain exemplary embodiments of the present invention will be more apparent from the following description taken in conjunction with the accompanying drawings in which:

FIG. 1 is a view illustrating a touch panel according to an exemplary embodiment of the present invention;

FIG. 2 is a view illustrating a touch panel that uses a Bio MEMS (Lab on a chip) technology according to an exemplary embodiment of the present invention;

FIG. 3 is a view illustrating a Bio MEMS (Lab on a chip) technology according to an exemplary embodiment of the present invention;

FIG. 4A is a first flowchart illustrating a process for operating a mobile terminal according to an exemplary embodiment of the present invention;

FIG. 4B is a second flowchart illustrating a process for operating a mobile terminal according to an exemplary embodiment of the present invention;

FIG. 5 is a flowchart illustrating a process for operating a server according to an exemplary embodiment of the present invention;

FIG. 6 is a flowchart illustrating a process for operating a hospital server according to an exemplary embodiment of the present invention; and

FIG. 7 is a view illustrating a block configuration of a mobile terminal and a network structure according to an exemplary embodiment of the present invention.

Throughout the drawings, like reference numerals will be understood to refer to like parts, components and structures.

### DETAILED DESCRIPTION OF EXEMPLARY EMBODIMENTS

The following description with reference to the accompanying drawings is provided to assist in a comprehensive understanding of exemplary embodiments of the invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. For the purposes of simplicity and to avoid redundancy, descriptions of well-known functions and constructions are omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

By the term “substantially” it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide.

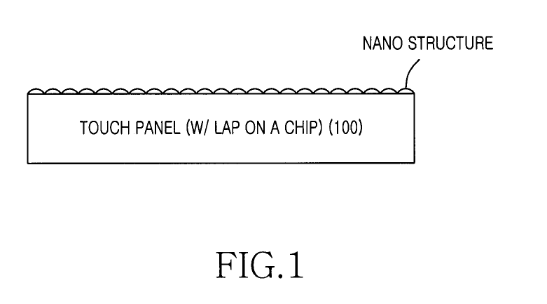

FIG. 1 is a view illustrating a touch panel according to an exemplary embodiment of the present invention.

Referring to FIG. 1, a nano technology that can collect body fluid generated from a finger or a hand, is applied to a window of the touch panel 100 according to an exemplary embodiment of the present invention.

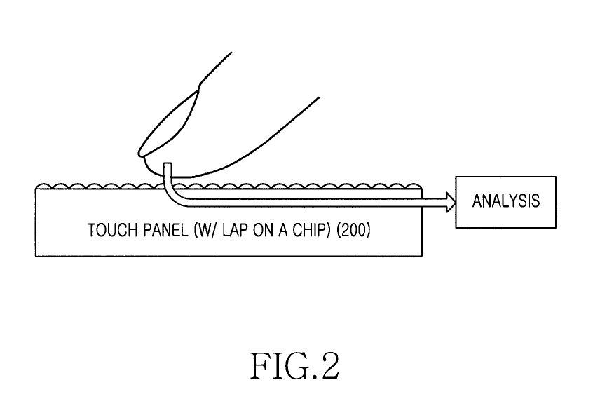

FIG. 2 is a view illustrating a touch panel that uses a Bio MEMS (i.e., Lab on a chip(LOC)) technology according to an exemplary embodiment of the present invention. Note that a Lab-on-a-chip (LOC) is a device that integrates one or more laboratory function on a single chip of only millimeters to a few square centimeters in size. LOC is a subset of MEMS devices.

Referring to FIG. 2, an exemplary embodiment of the present invention uses a Bio MEMS technology that can analyze body fluid collected via the window of the touch panel 200. According to an exemplary embodiment of the present invention, the mobile terminal analyzes body fluid which is left on the surface of the touch panel when a user touches or grips the touch panel of the mobile terminal using the Bio MEMS technology.

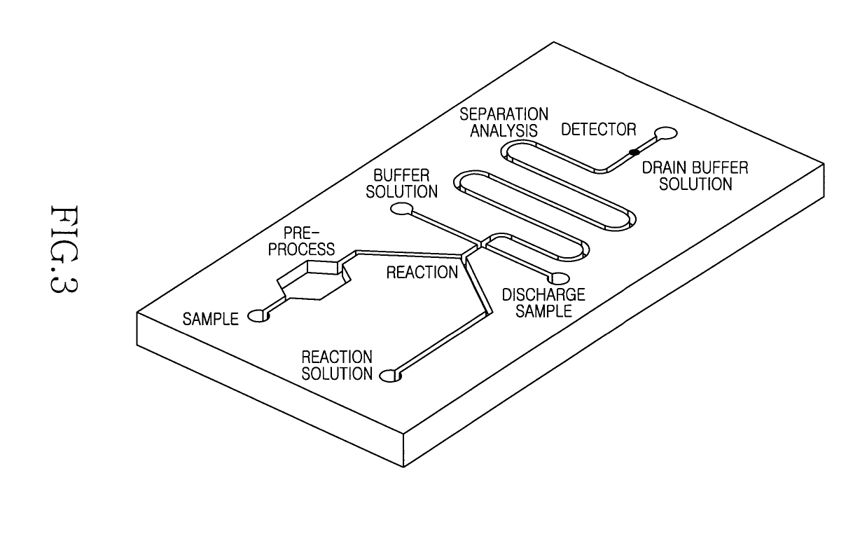

FIG. 3 is a view illustrating the configuration of a Bio MEMS (Lab on a chip) technology according to an exemplary embodiment of the present invention.

Referring to FIG. 3, the Bio MEMS (Lab on a chip) technology is a chemical micro processor that integrates various devices required for analysis on a chip having a size of several cm², made of glass, silicon, or plastic using a micro machining technology, and can perform an automated analysis at high speed, high efficiency, and low costs. This technology emerges as an important technology that can reduce costs and time required for new drug exploring in the field of a pharmaceutical industry that rapidly grows recently.

The Bio MEMS technology is a core platform technology that can be applied to various fields such as a medical diagnosis equipment, medical checkup at home or bedside, chemical or biological process monitoring, a portable environmental polluting material analyzer, a CBR unmanned chemical/living organism agent detection/identifying device as well as use for new drug exploring in a pharmaceutical industry.

An exemplary embodiment of the present invention applies the Bio MEMS technology to a window substrate by carving device portions on the window substrate using the Bio MEMS (Lab on a chip) technology as illustrated in the drawing.

An exemplary embodiment of the present invention may use a pressure sensor that can determine a pulse, a blood pressure, etc. The pressure sensor may be built in the window or positioned in a different portion of the mobile terminal other than the window.

In addition, an exemplary embodiment of the present invention may provide a health management system that can periodically transmit collected information to a server for update, and determine a user's health state in cooperation with a hospital based on the information stored in the server.

According to an exemplary embodiment of the present invention, a various types of user's body information may be stored in a server located in a network for a predetermined period, and when a change in the user's body information value occurs, a medical team in charge may notify the user when analysis is needed for update.

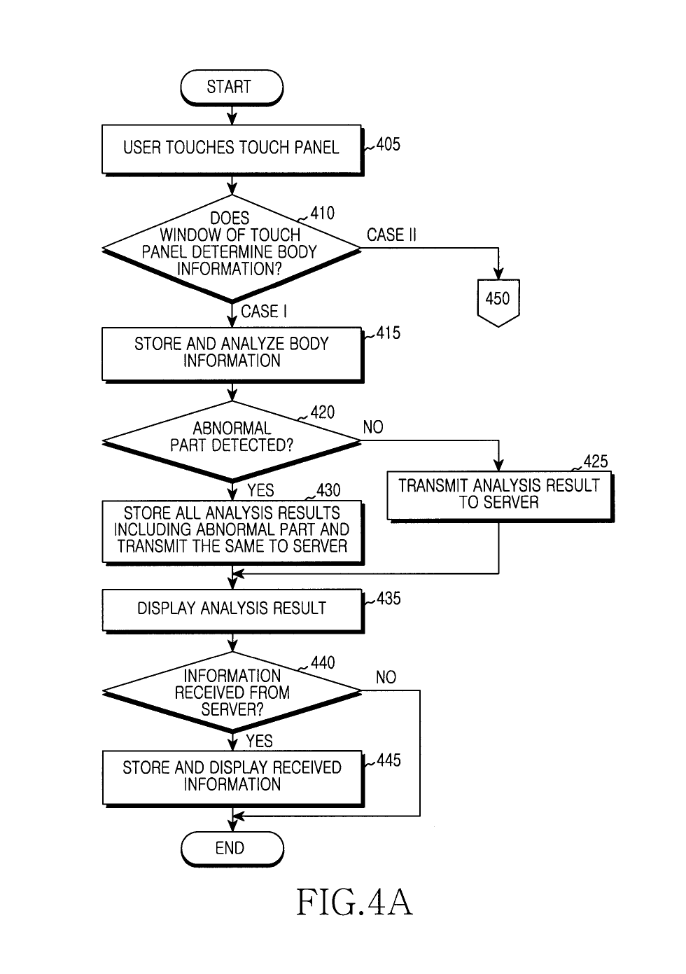

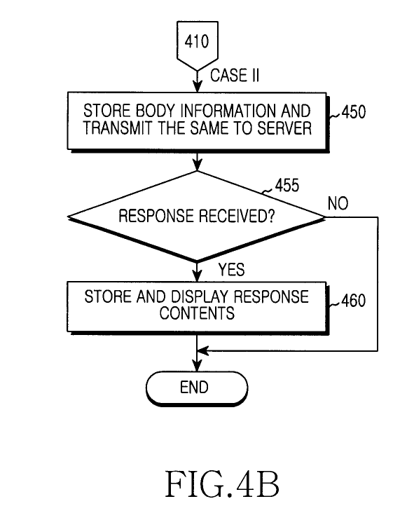

FIG. 4A is a first flowchart illustrating a process for operating a mobile terminal according to an exemplary embodiment of the present invention and FIG. 4B is a second flowchart illustrating a process for operating a mobile terminal according to an exemplary embodiment of the present invention.

Referring to FIG. 4A and FIG. 4B, during operation, a user touches a touch panel (step 405). When a window of the touch panel determines body information (step 410), the mobile terminal performs the following operations.

In case of Case I, the mobile terminal stores and analyzes the determined information (step 415).

When an abnormal part is detected as a result of the analysis by the mobile terminal (step 420), the mobile terminal stores all analysis results including the abnormal part and then transmits the same to a server (step 430), and then displays the analysis results (step 435).

Upon receiving information (response) from the server (step 440) relating to the detected result, the mobile terminal stores and displays the received information (step 445).

When the abnormal part is not detected as a result of the analysis by the mobile terminal (step 420), the mobile terminal transmits the analysis result to the server (step 425), and displays the analysis result (step 435).

In case of Case II, the mobile terminal stores determined body information and transmits the same to the server (step 450). Upon receiving a response from the server (step 455), the mobile terminal stores and displays the response contents (step 460).

The response contents may include a body information analysis result and a guide for the analysis result. In addition, the server may be a server that belongs to a network of a company to which the mobile terminal has subscribed.

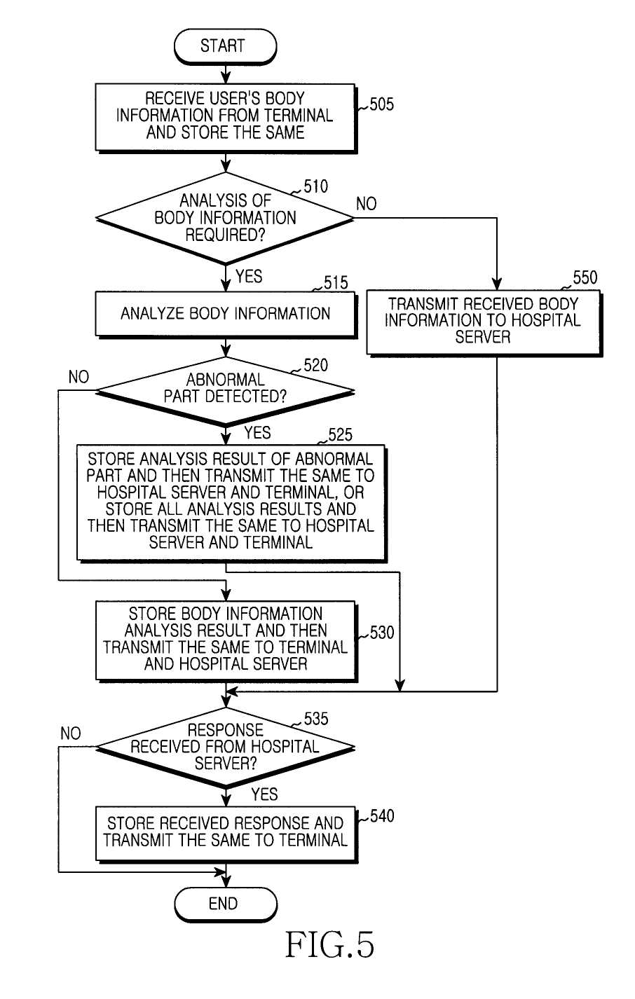

FIG. 5 is a flowchart illustrating a process for operating a server according to an exemplary embodiment of the present invention.

The server is a server of a company to which a mobile terminal has subscribed. The server may play a role of relaying between the mobile terminal and a hospital server, or analyze body information. The server may not be actually implemented depending on an implementation purpose.

Referring to FIG. 5, the server receives and stores body information from the mobile terminal (step 505).

In the case where analysis of the body information is not required, that is, the mobile terminal analyzes body information or the hospital server analyzes body information (step 510), the server transmits received body information to the hospital server (step 550). Here, the body information may include an analysis result where the body information has been analyzed by the mobile terminal.

Upon receiving a response from the hospital server (step 535), the server stores the received response and transmits the same to the mobile terminal (step 540).

In the case where the analysis of the body information is required (step 510), the server analyzes the body information (step 515).

When an abnormal part is detected as a result of the analysis (step 520), the server stores the analysis result regarding the abnormal part, and transmits the same to the hospital server and the mobile terminal, or stores all the analysis results and transmits the same to the hospital server and the mobile terminal (step 525).

When a response is received from the hospital server (step 535), the server stores the received response and transmits the same to the mobile terminal (step 540).

When the abnormal part is not detected (step 520), the server stores the body information analysis result and transmits the same to the mobile terminal and the hospital server (step 530).

Thereafter, when a response is received from the hospital server (step 535), the server stores the received response and transmits the same to the mobile terminal (step 540).

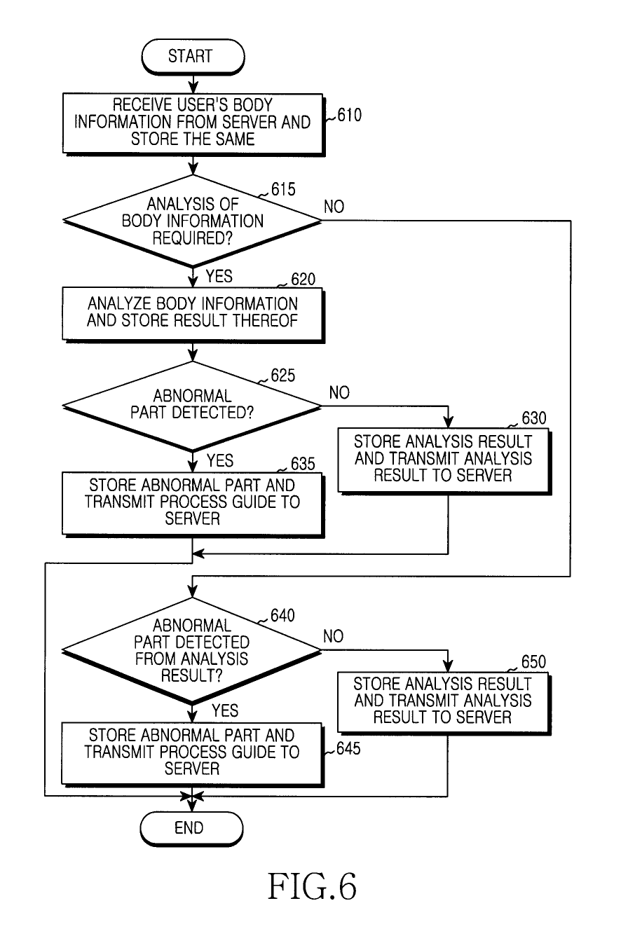

FIG. 6 is a flowchart illustrating a process for operating a hospital server according to an exemplary embodiment of the present invention.

Referring to FIG. 6, the hospital server receives and stores a user's body information (step 610). The body information may include analysis results where the body information has been analyzed by a mobile terminal or a server.

In the case where the analysis of the body information is not required (step 615), the hospital server determines whether an abnormal part is detected (step 640). This case is for supplementing the analysis of the body information by the server in FIG. 5 or the mobile terminal in FIG. 4, and may be omitted depending on necessity.

When the abnormal part is detected, the hospital server stores an analysis result regarding the abnormal part and transmits a guide thereof to the server (step 645).

When the abnormal part is not detected, the hospital server stores an analysis result and transmits the analysis result to the server (step 650).

In the case where the analysis of the body information is required (step 615), the hospital server analyzes the body information and stores a result thereof (step 620).

When an abnormal part is detected from an analysis result (step 625), the hospital server stores the analysis result regarding the abnormal part and transmits a guide thereof to the server (step 635).

When the abnormal part is not detected from the analysis result (step 625), the hospital server stores the analysis result and transmits the same to the server (step 630).

The above-described methods according to the present invention can be implemented in hardware, firmware or as software or computer code that can be stored in a recording medium such as a CD ROM, an RAM, a floppy disk, a hard disk, or a magneto-optical disk or downloaded over a network and stored on a non-transitory machine readable medium, so that the methods described herein can be rendered in such software using a general purpose computer, or a special processor or in programmable or dedicated hardware, such as an ASIC or FPGA. As would be understood in the art, the computer, the processor, microprocessor controller or the programmable hardware include memory components, e.g., RAM, ROM, Flash, etc. that may store or receive software or computer code that when accessed and executed by the computer, processor or hardware implement the processing methods described herein. In addition, it would be recognized that when a general purpose computer accesses code for implementing the processing shown herein, the execution of the code transforms the general purpose computer into a special purpose computer for executing the processing shown herein.

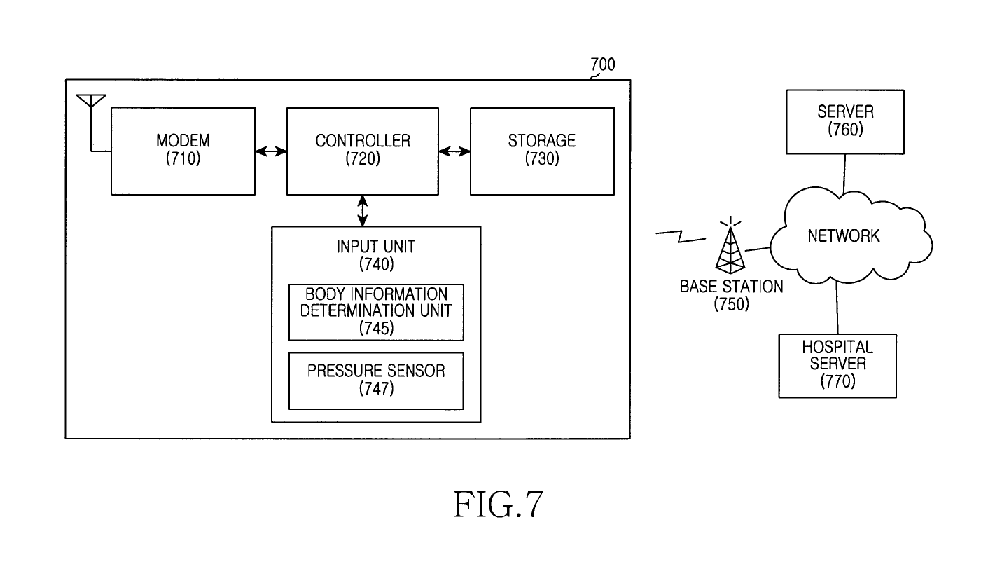

FIG. 7 is a view illustrating a block configuration of a mobile terminal and a network structure according to an exemplary embodiment of the present invention.

Referring to FIG. 7, the network includes a mobile terminal 700, a base station 750, a server 760, and a hospital server 770.

The mobile terminal includes a modem 710, a controller 720, a storage 730, and an input unit 740. The input unit 740 includes a body information determination unit 745 and a pressure sensor 747.

The modem 710 serves as a module for communicating with a different device, and may include a radio processor and a baseband processor. The radio processor converts a signal received via an antenna into a baseband signal to provide the same to the baseband processor, converts a baseband signal from the baseband processor into a Radio Frequency (RF) signal so that the RF signal may be transmitted on an RF path, and transmits the same via the antenna.

The controller 720 controls an overall operation of the mobile terminal. For example, the controller 720 performs processes and controls for voice communication and packet communication. More particularly, the controller 720 controls the input unit 740 to analyze body information determined by the input unit 740. Alternatively, the controller 720 displays information transmitted by the server 760 or the hospital server 770 via a display unit. Though not shown, the display unit may be positioned at the same position as the input unit 740 and may be realized as a touch screen.

The controller 720 transmits/receives information to/from the base station 750 via the modem 710 wirelessly, and the base station 750 transmits/receives wirelessly received information to/from the server 760 via a wired path.

The storage 730 stores programs for controlling an overall operation of the mobile terminal and temporary data occurring during an execution of programs. The storage 730 stores body information determined by the input unit 740 and stores information transmitted by the server 760 and the hospital server 770 under control of the controller 720.

The input unit 740 includes a touch panel including a window to which the Bio MEMS (lab on a chip) technology has been applied, and a pressure sensor 747 for determining a user's blood pressure. In FIG. 7, the window to which the Bio MEMS (lab on a chip) technology has been applied is denoted by a body information determination unit 745. The input unit 740 provides body information determined by the body information determination unit 745 and the pressure sensor 747 to the controller 720.

When a user touches the input unit 740, the controller 720 determines the user's body information.

When the controller 720 does not analyze the body information, the controller 720 stores the determined body information in the storage 730 and transmits the same to the server 760 via the modem 710.

Upon receiving a response from the server 760 via the modem 710, the controller 720 stores the response contents in the storage 730 and displays the same via a display unit. The response contents may include the body information analysis result and a guide corresponding to the analysis result.

In case of analyzing the body information, the controller 720 stores the determined body information in the storage 730 and analyzes the body information.

When an abnormal part is detected as a result of the analysis, the controller 720 stores all analysis results including the abnormal part in the storage 730, and transmits the same to the server 760 via the modem 710. In addition, the controller 720 displays the analysis result via the display unit.

When an abnormal part is not detected as a result of the analysis, the controller 720 transmits the analysis result to the server 760 via the modem 710 and displays the analysis result via the display unit.

When receiving information (response) from the server 760 via the modem 710, the controller 720 stores the received information in the storage 730 and displays the same via the display unit.

Though not shown, like the mobile terminal 700, the server 760 includes a wired modem, a controller, and a storage. The wired modem of the server 760 is responsible for encoding/decoding and modulation/demodulation of a transmission/reception signal for wired communication. The controller of the server 760 controls the modem and the storage of the server 760. The storage of the server 760 stores a program for controlling an overall operation of the server 760 and temporary data that occurs during execution of programs, and stores information transmitted by the mobile terminal 700 and the hospital server 770 under control of the controller of the server 760.

In the following, an expression that the controller of the server 760 stores denotes that the controller of the server 760 stores via the storage of the server 760. An expression that the controller of the server 760 transmits/receives information or a specific object denotes that the controller of the server 760 transmits/receives the information or the specific object via the wired modem of the server 760.

The controller of the server 760 receives and stores body information from the mobile terminal 700. In the case where analysis of the body information is not required, that is, the mobile terminal 700 analyzes the body information or the hospital server 770 analyzes the body information, the controller of the server 760 transmits received body information to the hospital server 770. Here, the body information may include an analysis result where the body information has been analyzed by the mobile terminal.

When receiving a response from the hospital server, the server 760 stores the received response and transmits the same to the mobile terminal 700.

When analysis of the body information is required, the server 760 analyzes the body information.

When an abnormal part is detected as a result of analysis, the server 760 stores an analysis result regarding the abnormal part and then transmits the same to the hospital server 770 and the mobile terminal 700, or stores all analysis results and then transmits the same to the hospital server 770 and the mobile terminal 700.

When the abnormal part is not detected as a result of analysis, the server 760 stores the body information analysis result and transmits the same to the mobile terminal 700 and the hospital server 770.

When receiving a response from the hospital server 770, the server 760 stores the received response and transmits the same to the mobile terminal 700.

Like the mobile terminal 700, the hospital server 770 includes a wired modem, a controller, and a storage. The wired modem of the hospital server 770 is responsible for encoding/decoding and modulation/demodulation of a transmission/reception signal for wired communication. The controller of the hospital server 770 controls the modem and the storage of the hospital server 770. The storage of the hospital server 770 stores a program for controlling an overall operation of the hospital server 770 and temporary data that occurs during execution of programs, and stores information transmitted by the mobile terminal 700 and the server 760 under control of the controller of the hospital server 770.

In the following description, an expression that the controller of the hospital server 770 stores denotes that the controller of the hospital server 770 stores via the storage of the hospital server 770. An expression that the controller of the hospital server 770 transmits/receives information or a specific object denotes that the controller of the hospital server 770 transmits/receives the information or the specific object via the wired modem of the hospital server 770.

The hospital server 770 receives and stores a user's body information. The body information may include an analysis result where the body information has been analyzed by the mobile terminal 700 or the server 760.

When analysis of the body information by the hospital server 770 is not required, the hospital server 770 determines whether an abnormal part is detected from the analysis result included in the body information.

When the abnormal part is detected, the hospital server 770 stores an analysis result for the abnormal part and transmits a guide thereof to the server 760.

When the abnormal part is not detected, the hospital server 770 stores the analysis result in the server 760.

When the analysis of the body information by the hospital server 770 is required, the hospital server 770 analyzes the body information and stores a result thereof.

When the abnormal part is detected from the analysis result of the hospital server 770, the hospital server 770 stores the analysis result regarding the abnormal part and transmits a guide thereof to the server 760.

When the abnormal part is not detected from the analysis result of the hospital server 770, the hospital server 770 stores the analysis result and transmits the same to the server 760.

Exemplary embodiments of the present invention have an advantage of managing a user's health on line by determining body information using a touch panel of a mobile terminal. In addition, the user has an advantage of managing his health by only touching a mobile terminal without visiting a hospital separately.

The above-described methods according to the present invention can be implemented in hardware, firmware or as software or computer code that can be stored in a recording medium such as a CD ROM, an RAM, a floppy disk, a hard disk, or a magneto-optical disk or downloaded over a network and stored on a non-transitory machine readable medium, so that the methods described herein can be rendered in such software using a general purpose computer, or a special processor or in programmable or dedicated hardware, such as an ASIC or FPGA. As would be understood in the art, the computer, the processor, microprocessor controller or the programmable hardware include memory components, e.g., RAM, ROM, Flash, etc. that may store or receive software or computer code that when accessed and executed by the computer, processor or hardware implement the processing methods described herein. In addition, it would be recognized that when a general purpose computer accesses code for implementing the processing shown herein, the execution of the code transforms the general purpose computer into a special purpose computer for executing the processing shown herein.

Although the invention has been shown and described with reference to certain exemplary embodiments thereof, it will be understood by those skilled in the art that various changes in form and details may be made therein without departing from the spirit and scope of the invention as defined by the appended claims and their equivalents. Therefore, the scope of the present invention should not be limited to the above-described embodiments but should be determined by not only the appended claims but also the equivalents thereof.

What is claimed is:

1. A method for determining a user's body information in a mobile terminal, the method comprising:
determining, by a touch panel, whether exists touch input is detected in the touch panel of the terminal having a Bio Micro Electric Mechanical System (MEMS) technology provided thereon;
upon detecting the touch input, determining, by a controller, the user's body information via the touch panel;
transmitting, by the controller, the determined body information to an upper node via a modem;
receiving, by the controller, body information analysis results from the upper node via the modem; and
displaying, by the controller, the body information analysis results via a display unit.

2. The method of claim 1, wherein the determining of the user's body information comprises:
analyzing the user's body fluid determined via a window inside the touch panel to which the Bio MEMS technology has been applied; and
determining the user's blood pressure or pulse using a pressure sensor inside the touch panel.

3. The method of claim 1, further comprising:
analyzing, by the controller, the determined body information;
displaying, by the controller, an analysis result;
transmitting, by the controller, an analysis result to the upper node; and
when a response to the analysis result is received from the upper node, displaying, by the controller, the response.

4. The method of claim 3, wherein the upper node comprises a server belonging to a company to which the mobile terminal has subscribed or a hospital server.

5. The method of claim 1, further comprising storing, by the controller, the body information and information for communicating with the upper node to a storage.

6. An apparatus of a mobile terminal that determines a user's body information, comprising:
a touch panel having a window to which a Bio Micro Electric Mechanical System (MEMS) technology has been applied and detecting the user's touch;
a modem for transmitting/receiving information to/from an upper node;
a controller for, upon detecting a touch input on the touch panel, determining the user's body information via the touch panel, transmitting the determined body information to the upper node via the modem, and receiving body information analysis results from the upper node via the modem; and
a display unit for displaying the body information analysis results.

7. The apparatus of claim 6, further comprising a pressure sensor.

8. The apparatus of claim 7, wherein the controller analyzes the user's body fluid determined via a window inside the touch panel to which the Bio MEMS technology has been applied, and determines the user's blood pressure or pulse using a pressure sensor inside the touch panel.

9. The apparatus of claim 6, wherein the controller analyzes the determined body information, displays an analysis result via the display unit, transmits the analysis result to the upper node via the modem, and upon receiving a response to the analysis result from the upper node via the modem, displays the response via the display unit.

10. The apparatus of claim 8, wherein the upper node comprises a server belonging to a company to which the mobile terminal has subscribed or a hospital server.

11. The apparatus of claim 6, further comprising a storage for storing the body information and information for the controller to communicate with the upper node.

12. A mobile communication system for performing a user's health management, comprising:
a mobile terminal for determining whether a touch input is detected on a touch panel to which a Bio Micro Electric Mechanical System (MEMS) technology has been applied, and if so, determining the user's body information, transmitting the determined body information to a server, and upon receiving a response from the server, displaying the response;
the server for transmitting the body information transmitted by the mobile terminal to a hospital server, and transmitting a response transmitted by the hospital server to the mobile terminal; and
the hospital server for analyzing the user's body information transmitted by the server, and when an abnormal part exists as a result of the body information analysis, transmitting an analysis result comprising the abnormal part and a processing guide for the abnormal part to the server.

13. The system of claim 12, wherein the mobile terminal analyzes the determined user's body fluid via a window inside the touch panel to which the Bio MEMS technology has been applied, and determines the user's blood pressure or pulse using a pressure sensor inside the touch panel.

14. The system of claim 12, wherein the mobile terminal analyzes the determined body information, displays an analysis result, transmits the analysis result to the server, and when receiving a response to the analysis result from the server, displays the response.

15. The system of claim 12, wherein the mobile terminal, the server, and the hospital server store the body information and information for communicating with a different node.

16. The system of claim 11, wherein the server analyzes the received body information, transmits an analysis result to the mobile terminal and the hospital server, and when receiving a response to the analysis result from the hospital server, transmits the response to the mobile terminal.

17. The system of claim 12, wherein when an abnormal part exists in the received analysis result, the hospital server transmits a processing guide for the abnormal part to the server.
